SEQUENCE LISTING (8 sequences)

SEQ ID NO: 1 - protein, 32 aa, Artificial sequence
  Synthetic sequence VPAC2 receptor peptide agonist
XXXXXXTXXXTXXXXXXXXXXXXXXXXXXXXX

SEQ ID NO: 2 - protein, 32 aa, Artificial sequence
  Synthetic sequence VPAC2 receptor peptide agonist
HSXAVFTXXXTXXXXXXXXXXXXXXXXXXXXX

SEQ ID NO: 3 - protein, 28 aa, Homo sapiens
HSDAVFTDNYTRLRKQMAVKKYLNSILN

SEQ ID NO: 4 - protein, 27 aa, Homo sapiens
  MOD_RES (27)..(27): AMIDATION
HSDGIFTDSYSRYRKQMAVKKYLAAVL

SEQ ID NO: 5 - protein, 37 aa, Homo sapiens
  MOD_RES (37)..(37): AMIDATION
HSDGIFTDSYSRYRKQMAVKKYLAAVLGKRYQRVKNK

SEQ ID NO: 6 - protein, 9 aa, Artificial sequence
  Synthetic sequence N-terminal modification to a VPAC2 receptor agonist wherein the Arg at position 9 is linked to the N-terminus of the peptide agonist
SWCEPGWCR

SEQ ID NO: 7 - protein, 39 aa, Artificial sequence
  Synthetic sequence VPAC2 receptor peptide agonist P603
HSDAVFTEQYTXLRXQLAXXXYXQXIXXGGPSSGAPPPK

SEQ ID NO: 8 - protein, 11 aa, Artificial sequence
  Synthetic sequence C-terminal extension of a VPAC2 receptor peptide agonist
GGPSSGAPPPK

(19) United States
(12) Patent Application Publication
(10) Pub. No.: US 2009/0082276 A1
(43) Pub. Date: Mar. 26, 2009

(54) SELECTIVE VPAC2 RECEPTOR PEPTIDE AGONISTS

(76) Inventors: Lianshan Zhang, Carmel, IN (US); Jorge Alsina-Fernandez, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 12/278,337

(22) PCT Filed: Feb. 20, 2007
(86) PCT No.: PCT/US2007/062414

§ 371 Date: Aug. 5, 2008

Related U.S. Application Data
(60) Provisional application No. 60/743,366, filed on Feb. 28, 2006.

(51) Int. Cl. A61K 38/16; C07K 14/00

(52) U.S. Cl. 514/12; 530/324; 530/317

(57) ABSTRACT

The present invention encompasses peptides that selectively activate the VPAC2 receptor and are useful in the treatment of diabetes.

The present invention relates to selective VPAC2 receptor peptide agonists.

In particular, the present invention relates to selective VPAC2 receptor peptide agonists comprising a C-terminal extension, which comprises the amino acid sequence: GGPSSGAPPPK(E-C₁₆).

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications, however, do not slow the progressive loss of β-cell function that occurs in NIDDM patients and, thus, are not sufficient to control blood glucose levels in the later stages of the disease. Also, treatment with currently available medications exposes NIDDM patients to potential side effects such as hypoglycemia, gastrointestinal problems, fluid retention, oedema, and/or weight gain.

Pituitary adenylate cyclase-activating peptide (PACAP) and vasoactive intestinal peptide (VIP) belong to the same family of peptides as secretin and glucagon. PACAP and VIP work through three G-protein-coupled receptors that exert their action through the cAMP-mediated and other Ca²⁺-mediated signal transduction pathways. These receptors are known as the PACAP-preferring type 1 (PAC1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Commun., 193:546-553 (1993); Lutz, et al., FEBS Lett., 458: 197-203 (1999); Adamou, et al., Biochem. Biophys. Res. Commun., 209: 385-392 (1995)). A series of PACAP analogues is disclosed in U.S. Pat. No. 6,242,563 and WO 2000/05260.

PACAP has comparable activities towards all three receptors, whilst VIP selectively activates the two VPAC receptors (Tsutsumi et al., Diabetes, 51:1453-1460 (2002)). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also to increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). Furthermore, it appears that VIP-induced watery diarrhoea in rats is mediated by only one of the VPAC receptors, VPAC1 (Ito et al., Peptides, 22:1139-1151 (2001); Tsutsumi et al., Diabetes, 51:1453-1460 (2002)). In addition, the VPAC1 and PAC1 receptors are expressed on α-cells and hepatocytes and, thus, are most likely involved in the effects on hepatic glucose output.

Exendin-4 is found in the salivary excretions from the Gila Monster, Heloderma Suspectum, (Eng et al., J. Biol. Chem., 267(11):7402-7405 (1992)). It is a 39 amino acid peptide, which has glucose dependent insulin secretagogue activity. Particular PEGylated Exendin and Exendin agonist peptides are described in WO 2000/66629. Exendin derivatives, which have at least one amino acid which is attached to a lipophilic substituent, are described in WO 99/43708.

Recent studies have shown that peptides selective for the VPAC2 receptor are able to stimulate insulin secretion from the pancreas without gastrointestinal (GI) side effects and without enhancing glucagon release and hepatic glucose output (Tsutsumi et al., Diabetes, 51:1453-1460 (2002)). Peptides selective for the VPAC2 receptor, were initially identified by modifying VIP and/or PACAP (See, for example, Xia et al., J Pharmacol Exp Ther., 281:629-633 (1997); Tsutsumi et al., Diabetes, 51:1453-1460 (2002); WO 01/23420; WO 2004/006839).

Many of the VPAC2 receptor peptide agonists reported to date have, however, less than desirable potency, selectivity, and stability profiles, which could impede their clinical viability. In addition, many of these peptides are not suitable for commercial candidates as a result of stability issues associated with the polypeptides in formulation, as well as issues with the short half-life of these polypeptides in vivo. It has, furthermore, been identified that some VPAC2 receptor peptide agonists are inactivated by dipeptidyl-peptidase (DPP-IV). A short serum half-life could hinder the use of these agonists as therapeutic agents. There is, therefore, a need for new therapies, which overcome the problems associated with current medications for NIDDM.

The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists.

According to a first aspect of the invention, there is provided a VPAC2 receptor peptide agonist comprising a sequence of the formula:

(SEQ ID NO: 1)

Xaa₁-Xaa₂₋Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂
Formula 1

wherein:
Xaa₁ is: His, dH, or is absent;

### Xaa₂ is: dA, Ser, Val, Gly, Thr, Leu, dS, Pro, or Aib;

### Xaa₃ is: Asp or Glu;

### Xaa₄ is: Ala, Ile, Tyr, Phe, Val, Thr, Leu, Trp, Gly, dA, Aib, or NMeA;

### Xaa₅ is: Val, Leu, Phe, Ile, Thr, Trp, Tyr, dV, Aib, or NMeV;

### Xaa₆ is: Phe, Ile, Leu, Thr, Val, Trp, or Tyr;

### Xaa₈ is: Asp, Glu, Ala, Lys, Leu, Arg, or Tyr;

### Xaa₉ is: Asn, Gln, Glu, Ser, Cys, or K(CO(CH₂)₂SH);

### Xaa₁₀ is: Tyr, Trp, or Tyr(OMe);

### Xaa₁₂ is: Arg, Lys, hR, Orn, Aib, Ala, Leu, Gln, Phe, or Cys;

### Xaa₁₃ is: Leu, Phe, Glu, Ala, Aib, Ser, Cys, or K(CO(CH₂)₂SH);

### Xaa₁₄ is: Arg, Leu, Lys, Ala, hR, Orn, Phe, Gln, Aib, or Cit;

### Xaa₁₅ is: Lys, Ala, Arg, Glu, Leu, Orn, Phe, Gln, Aib, K(Ac), Cys, K(W), or K(CO(CH₂)₂SH);

### Xaa₁₆ is: Gln, Lys, Ala, Ser, Cys, or K(CO(CH₂)₂SH);

### Xaa₁₇ is: Val, Ala, Leu, Ile, Met, Nle, Lys, Aib, Ser, Cys, K(CO(CH₂)₂SH), or K(W);

### Xaa₁₈ is: Ala, Ser, Cys, or Abu;

### Xaa₁₉ is: Ala, Leu, Gly, Ser, Cys, K(CO(CH₂)₂SH), or Abu;

### Xaa₂₀ is: Lys, Gln, hR, Arg, Ser, Orn, Ala, Aib, Trp, Thr, Leu, Ile, Phe, Tyr, Val, K(Ac), Cys, or K(CO(CH₂)₂SH);

### Xaa₂₁ is: Lys, Arg, Ala, Phe, Aib, Leu, Gln, Orn, hR, K(Ac), Ser, Cys, K(W), K(CO(CH₂)₂SH), or hC;

### Xaa₂₂ is: Tyr, Trp, Phe, Thr, Leu, Ile, Val, Tyr(OMe), Ala, Aib, or Ser;

### Xaa₂₃ is: Leu, Phe, Ile, Ala, Trp, Thr, Val, Aib, or Ser;

### Xaa₂₄ is: Gln, Asn, Ser, Cys, K(CO(CH₂)₂SH), or K(W);

### Xaa₂₅ is: Ser, Asp, Phe, Ile, Leu, Thr, Val, Trp, Gln, Asn, Tyr, Aib, Glu, Cys, K(CO(CH₂)₂SH), or hC;

### Xaa₂₆ is: Ile, Leu, Thr, Val, Trp, Tyr, Phe, Aib, Ser, Cys, K(CO(CH₂)₂SH), or K(W);

### Xaa₂₇ is: Lys, hR, Arg, Gln, Orn, or dK;

### Xaa₂₈ is: Asn, Gln, Lys, Arg, Aib, Orn, hR, Pro, dK, Cys, K(CO(CH₂)₂SH), or K(W);

Xaa₂₉ is: Lys, Ser, Arg, Asn, hR, Cys, Orn, or is absent;
Xaa₃₀ is: Arg, Lys, Ile, hR, or is absent;
Xaa₃₁ is: Tyr, His, Phe, Gln, or is absent; and
Xaa₃₂ is: Cys, or is absent;
provided that if Xaa₂₉, Xaa₃₀, Xaa₃₁, or Xaa₃₂ is absent, the next amino acid present downstream is the next amino acid in the peptide agonist sequence; and a C-terminal extension comprising the amino acid sequence:

| | |
|---|---|
| GGPSSGAPPPK(E-C₁₆) | (SEQ ID NO: 8) |

wherein the C-terminal amino acid may be amidated.

Preferably, the VPAC2 receptor peptide agonist comprises a sequence of the formula:

(SEQ ID NO: 2)

His-Ser-Xaa₃-Ala-Val-Phe-Thr-Xaa₈-Xaa₉-Xaa₁₀-Thr-
Xaa₁₂-Xaa₁₃-Xaa₁₄-Xaa₁₅-Xaa₁₆-Xaa₁₇-Xaa₁₈-Xaa₁₉-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂
Formula 2

wherein:

### Xaa₃ is: Asp or Glu;

### Xaa₈ is: Asp, Glu, Ala, Lys, Leu, Arg, or Tyr;

### Xaa₉ is: Asn, Gln, Glu, Ser, Cys, or K(CO(CH₂)₂SH);

### Xaa₁₀ is: Tyr, Trp, or Tyr(OMe);

### Xaa₁₂ is: Arg, Lys, hR, Orn, Aib, Ala, Leu, Gln, Phe, or Cys;

### Xaa₁₃ is: Leu, Phe, Glu, Ala, Aib, Ser, Cys, or K(CO(CH₂)₂SH);

### Xaa₁₄ is: Arg, Leu, Lys, Ala, hR, Orn, Phe, Gln, Aib, or Cit;

### Xaa₁₅ is: Lys, Ala, Arg, Glu, Leu, Orn, Phe, Gln, Aib, K(Ac), Cys, K(W), or K(CO(CH₂)₂SH);

### Xaa₁₆ is: Gln, Lys, Ala, Ser, Cys, or K(CO(CH₂)₂SH);

### Xaa₁₇ is: Val, Ala, Leu, Ile, Met, Nle, Lys, Aib, Ser, Cys, K(CO(CH₂)₂SH), or K(W);

### Xaa₁₈ is: Ala, Ser, Cys, or Abu;

### Xaa₁₉ is: Ala, Leu, Gly, Ser, Cys, K(CO(CH₂)₂SH), or Abu;

### Xaa₂₀ is: Lys, Gln, hR, Arg, Ser, Orn, Ala, Aib, Trp, Thr, Leu, Ile, Phe, Tyr, Val, K(Ac), Cys, or K(CO(CH₂)₂SH);

### Xaa₂₁ is: Lys, Arg, Ala, Phe, Aib, Leu, Gln, Orn, hR, K(Ac), Ser, Cys, K(W), K(CO(CH₂)₂SH), or hC;

### Xaa₂₂ is: Tyr, Trp, Phe, Thr, Leu, Ile, Val, Tyr(OMe), Ala, Aib, or Ser;

### Xaa₂₃ is: Leu, Phe, Ile, Ala, Trp, Thr, Val, Aib, or Ser;

### Xaa₂₄ is: Gln, Asn, Ser, Cys, K(CO(CH₂)₂SH), or K(W);

### Xaa₂₅ is: Ser, Asp, Phe, Ile, Leu, Thr, Val, Trp, Gln, Asn, Tyr, Aib, Glu, Cys, K(CO(CH₂)₂SH), or hC;

### Xaa₂₆ is: Ile, Leu, Thr, Val, Trp, Tyr, Phe, Aib, Ser, Cys, K(CO(CH₂)₂SH), or K(W);

### Xaa₂₇ is: Lys, hR, Arg, Gln, Orn, or dK;

### Xaa₂₈ is: Asn, Gln, Lys, Arg, Aib, Orn, hR, Pro, dK, Cys, K(CO(CH₂)₂SH), or K(W);

Xaa₂₉ is: Lys, Ser, Arg, Asn, hR, Cys, Orn, or is absent;
Xaa₃₀ is: Arg, Lys, Ile, hR, or is absent;
Xaa₃₁ is: Tyr, His, Phe, Gln, or is absent; and
Xaa₃₂ is: Cys, or is absent;
provided that if Xaa₂₉, Xaa₃₀, Xaa₃₁, or Xaa₃₂ is absent, the next amino acid present downstream is the next amino acid in the peptide agonist sequence; and a C-terminal extension comprising the amino acid sequence:

| | |
|---|---|
| GGPSSGAPPPK(E-C₁₆) | (SEQ ID NO: 8) |

wherein the C-terminal amino acid may be amidated.

Preferably, the VPAC2 receptor peptide agonist of the present invention comprises a sequence of Formula 1 (SEQ ID NO: 1) or Formula 2 (SEQ ID NO: 2) wherein Xaa₃ is Asp or Glu, Xaa₈ is Asp or Glu, Xaa₉ is Asn or Gln, Xaa₁₀ is Tyr or Tyr(OMe), Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, Orn, or Arg, Xaa₁₆ is Gln or Lys, Xaa₁₇ is Val, Leu, Ala, Ile, Lys, or Nle, Xaa₁₉ is Ala or Abu, Xaa₂₀ is Lys, Val, Leu, Aib, Ala, Gln, or Arg, Xaa₂₁ is Lys, Aib, Orn, Ala, Gln, or Arg, Xaa₂₃ is Leu or Aib, Xaa₂₅ is Ser or Aib, Xaa₂₇ is Lys, Orn, hR, or Arg, Xaa₂₈ is Asn, Gln, Lys, hR, Aib, Orn, or Pro and/or Xaa₂₉ is Lys, Orn, hR, or is absent.

Preferably, the VPAC2 receptor peptide agonist of the present invention comprises a sequence of Formula 1 (SEQ ID NO: 1) or Formula 2 (SEQ ID NO: 2) wherein Xaa₈ is Glu, Xaa₉ is Gln, and Xaa₁₀ is Tyr(OMe).

Preferably, the VPAC2 receptor peptide agonist of the present invention comprises a sequence of Formula 1 (SEQ ID NO: 1) or Formula 2 (SEQ ID NO: 2) wherein either Xaa₁₄ or Xaa₁₅ is Aib.

Preferably, the VPAC2 receptor peptide agonist of the present invention comprises a sequence of Formula 1 (SEQ ID NO: 1) or Formula 2 (SEQ ID NO: 2) wherein either Xaa₂₀ or Xaa₂₁ is Aib.

More preferably, the VPAC2 receptor peptide agonist of the present invention comprises a sequence of Formula 1 (SEQ ID NO: 1) or Formula 2 (SEQ ID NO: 2) wherein Xaa₁₅ is Aib and/or Xaa₂₀ is Aib.

Preferably, the VPAC2 receptor peptide agonist of the present invention comprises a sequence of Formula 1 (SEQ ID NO: 1) or Formula 2 (SEQ ID NO: 2) wherein Xaa₁₂, Xaa₂₁, Xaa₂₇ and Xaa₂₈ are all Orn.

Preferably, the VPAC2 receptor peptide agonist of the present invention comprises a sequence of Formula 1 (SEQ ID NO: 1) or Formula 2 (SEQ ID NO: 2) wherein Xaa₁₉ is Abu.

Preferably, the VPAC2 receptor peptide agonist of the present invention comprises a sequence of Formula 1 (SEQ ID NO: 1) or Formula 2 (SEQ ID NO: 2) wherein Xaa₂₃ is Aib.

Preferably, the VPAC2 receptor peptide agonist of the present invention comprises a sequence of Formula 1 (SEQ ID NO: 1) or Formula 2 (SEQ ID NO: 2) wherein Xaa₂₅ is Aib.

Preferably, the VPAC2 receptor peptide agonist of the present invention comprises a sequence of Formula 1 (SEQ ID NO: 1) or Formula 2 (SEQ ID NO: 2) wherein Xaa₃₀, Xaa₃₁ and Xaa₃₂ are absent. Even more preferably Xaa₂₉, Xaa₃₀, Xaa₃₁ and Xaa₃₂ are all absent.

Preferably, the VPAC2 receptor peptide agonist of the present invention is PEGylated.

A PEG molecule(s) may be covalently attached to any Lys, Cys, K(W) or K(CO(CH₂)₂SH) residue(s) at any position in the VPAC2 receptor peptide agonist according to the first aspect of the present invention.

Any Lys residue in the VPAC2 receptor peptide agonist may be substituted for a K(W) or a K(CO(CH₂)₂SH), which may be PEGylated. In addition, any Cys residue in the peptide agonist may be substituted for a modified cysteine residue, for example, hC. The modified Cys residue may be covalently attached to a PEG molecule.

Where there is more than one PEG molecule, there may be a combination of Lys, Cys, K(CO(CH₂)₂SH) and K(W) PEGylation. For example, if there are two PEG molecules, one may be attached to a Lys residue and one may be attached to a Cys residue.

Preferably, the PEG molecule is branched. Alternatively, the PEG molecule may be linear.

Preferably, the PEG molecule is between 1,000 daltons and 100,000 daltons in molecular weight. More preferably the PEG molecule is selected from 10,000, 20,000, 30,000, 40,000, 50,000 and 60,000 daltons. Even more preferably, it is selected from 20,000, 30,000, 40,000, or 60,000 daltons. Where there are two PEG molecules covalently attached to the peptide agonist of the present invention, each is 1,000 to 40,000 daltons and preferably, they have molecular weights of 20,000 and 20,000 daltons, 10,000 and 30,000 daltons, 30,000 and 30,000 daltons, or 20,000 and 40,000 daltons.

Preferably, the VPAC2 receptor peptide agonist of the present invention is cyclic.

The VPAC2 receptor peptide agonist may be cyclised by means of a lactam bridge. It is preferred that the lactam bridge is formed by the covalent attachment of the side chain of the residue at Xaaₙ to the side chain of the residue at Xaaₙ₊₄, wherein n is 1 to 28. Preferably, n is 12, 20, or 21. More preferably, n is 21. It is also preferred that the lactam bridge is formed by the covalent attachment of the side chain of a Lys or Orn residue to the side chain of an Asp or Glu residue. A Lys or Orn residue may be substituted for a Dab residue, the side chain of which may be covalently attached to the side chain of an Asp or Glu residue.

The VPAC2 receptor peptide agonist may alternatively be cyclised by means of a disulfide bridge. It is preferred that the disulfide bridge is formed by the covalent attachment of the side chain of the residue at Xaaₙ to the side chain of the residue at Xaaₙ₊₄, wherein n is 1 to 28. Preferably, n is 12, 20, or 21. More preferably, n is 21. It is also preferred that the disulfide bridge is formed by the covalent attachment of the side chain of a Cys or hC residue to the side chain of another Cys or hC residue.

Alternatively, the lactam bridge or the disulfide bridge may be formed by the covalent attachment of the side chain of the residue at Xaaₙ to the side chain of the residue at Xaaₙ₊₃, wherein n is 1 to 28. The lactam bridge or the disulfide bridge may also be formed by the covalent attachment of the side chain of the residue at Xaaᵢ to the side chain of the residue at Xaaᵢ₊₇ or Xaaᵢ₊₈, wherein i is 1 to 24.

The VPAC2 receptor peptide agonist sequence may further comprise a histidine residue at the N-terminus of the peptide before Xaa₁.

Preferably, the VPAC2 receptor peptide agonist according to the first aspect of the present invention further comprises a N-terminal modification at the N-terminus of the peptide agonist wherein the N-terminal modification is selected from:
(a) addition of D-histidine, isoleucine, methionine, or norleucine;
(b) addition of a peptide comprising the sequence Ser-Trp-Cys-Glu-Pro-Gly-Trp-Cys-Arg (SEQ ID NO: 6) wherein the Arg is linked to the N-terminus of the peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; an aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; an arylC₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or arylC₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃, arylC₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, arylC₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

Preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. It is especially preferred that the N-terminal modification is the addition of acetyl or hexanoyl.

It will be appreciated by the person skilled in the art that VPAC2 receptor peptide agonists comprising various combinations of peptide sequence according to Formula 1 or Formula 2 and N-terminal modifications as described herein, may be made based on the above disclosure.

It is preferred that the VPAC2 receptor peptide agonist according to the first aspect of the present invention comprises the amino acid sequence:

| | SEQ | |
|---|---|---|
| | ID | |
| Agonist | NO | Sequence |
| P603 | 7 | C6-HSDAVFTEQY(OMe)TOrnLRAibQLAAbuAibOrn |
| | | YAibQAibIOrnOrnGGPSSGAPPPK(E-C16)-NH₂ |

According to the second aspect of the present invention, there is provided a pharmaceutical composition comprising a cyclic VPAC2 receptor peptide agonist for the present invention and one or more pharmaceutically acceptable diluents, carriers and/or excipients.

According to a third aspect of the present invention, there is provided a VPAC2 receptor peptide agonist of the present invention for use as a medicament.

According to a fourth aspect of the present invention, there is provided a VPAC2 receptor peptide agonist of the present invention for use in the treatment of non-insulin-dependent diabetes or insulin-dependent diabetes, or for use in the suppression of food intake.

According to a fifth aspect of the present invention, there is provided the use of a VPAC2 receptor peptide agonist of the present invention for the manufacture of a medicament for the treatment of non-insulin-dependent diabetes, or insulin-dependent diabetes, or for the suppression of food intake.

According to a further aspect of the present invention, there is provided a method of treating non-insulin-dependent diabetes or insulin-dependent diabetes, or of suppressing food intake in a patient in need thereof comprising administering an effective amount of a VPAC2 receptor peptide agonist of the present invention.

According to yet a further aspect of the present invention, there is provided a pharmaceutical composition containing a VPAC2 receptor peptide agonist of the present invention for treating non-insulin-dependent diabetes or insulin-dependent diabetes, or for suppressing food intake.

The VPAC2 receptor peptide agonists of the present invention have the advantage that they have enhanced selectivity, potency and/or stability over known VPAC2 receptor peptide agonists. In vivo the palmitic acid group at the C-terminus may bind to serum albumin, thereby preventing kidney filtration and prolonging the biological action of the VPAC2 receptor peptide agonist.

The VPAC2 receptor peptide agonists of the present invention may be PEGylated.

The covalent attachment of one or more molecules of PEG to particular residues of a VPAC2 receptor peptide agonist results in a biologically active, PEGylated VPAC2 receptor peptide agonist with an extended half-life and reduced clearance when compared to that of non-PEGylated VPAC2 receptor peptide agonists.

The VPAC2 receptor peptide agonists of the present invention may be cyclic.

Cyclic VPAC2 receptor peptide agonists have restricted conformational mobility compared to linear VPAC2 peptide receptor agonists of small/medium size and for this reason cyclic peptides have a smaller number of allowed conformations compared with linear peptides. Constraining the conformational flexibility of linear peptides by cyclisation enhances receptor-binding affinity, increases selectivity and improves proteolytic stability and bioavailability compared with linear peptides.

The term “VPAC2” is used to refer to the particular receptor (Lutz, et al., FEBS Lett., 458: 197-203 (1999); Adamou, et al., Biochem. Biophys. Res. Commun., 209: 385-392 (1995)) that the agonists of the present invention activate. This term also is used to refer to the agonists of the present invention.

A “selective VPAC2 receptor peptide agonist” or a “VPAC2 receptor peptide agonist” of the present invention is a peptide that selectively activates the VPAC2 receptor to induce insulin secretion. Preferably, the sequence for a selective VPAC2 receptor peptide agonist of the present invention has twenty-eight to thirty-two naturally occurring and/or non-naturally occurring amino acids and additionally comprises a C-terminal extension, comprising the amino acid sequence: GGPSSGAPPPK (E-C₁₆).

A “selective PEGylated VPAC2 receptor peptide agonist” or “PEGylated VPAC2 receptor peptide agonist” is a selective VPAC2 receptor peptide agonist covalently attached to one or more molecules of polyethylene glycol (PEG), or a derivative thereof, wherein each PEG is attached to a cysteine or lysine amino acid, or to a K(W) or K(CO(CH₂)₂SH) residue.

A “selective cyclic VPAC2 receptor peptide agonist” or a “cyclic VPAC2 receptor peptide agonist” is a selective VPAC2 receptor peptide agonist cyclised by means of a covalent bond linking the side chains of two amino acids in the peptide chain. The covalent bond may, for example, be a lactam bridge or a disulfide bridge.

Selective VPAC2 receptor peptide agonists of the present invention have a C-terminal extension. The “C-terminal extension” of the present invention comprises the sequence GGPSSGAPPPK (E-C₁₆) and is linked to the C-terminus of the peptide sequence of Formula 1 (SEQ ID NO: 1) or Formula 2 (SEQ ID NO: 2) at the N-terminus of the C-terminal extension via a peptide bond. The sequence GGPSSGAPPPK(E-C16) is a variant of the C-terminal sequence of Exendin-4. The C-terminal lysine residue has a glutamic acid residue, which is acylated at the alpha-amino group with palmitic acid, attached to its side chain.

As used herein, the term “linked to” with reference to the term C-terminal extension, includes the addition or attachment of amino acids or chemical groups directly to the C-terminus of the peptide sequence of Formula 1 or Formula 2.

Optionally, the selective VPAC2 receptor peptide agonist may also have an N-terminal modification. The term “N-terminal modification” as used herein includes the addition or attachment of amino acids or chemical groups directly to the N-terminus of a peptide and the formation of chemical groups, which incorporate the nitrogen at the N-terminus of a peptide.

The N-terminal modification may comprise the addition of one or more naturally occurring or non-naturally occurring amino acids to the VPAC2 receptor peptide agonist sequence, preferably there are not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: SEQ ID NO: 6 Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the VPAC2 receptor agonist. The addition of the above N-terminal modifications may be achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 2004/006839) or a carbamate group at the N-terminus. Also, the N-terminus may be modified by the addition of pyroglutamic acid, or 6-aminohexanoic acid.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminus of the peptide.

The N-terminus may alternatively be modified by the addition of methionine sulfoxide, biotinyl-6-aminohexanoic acid, or —C(═NH)—NH₂. The addition of —C(═NH)—NH₂ is a guanidation modification, where the terminal NH₂ of the N-terminal amino acid becomes —NH—C(═NH)—NH₂.

Most of the sequences of the present invention, including the N-terminal modifications and the C-terminal extensions contain the standard single letter or three letter codes for the twenty naturally occurring amino acids. The other codes used are defined as follows:
Ac=acetyl
C6=hexanoyl
d=the D isoform (nonnaturally occurring) of the respective amino acid, e.g., dA=D-alanine, dS=D-serine, dK=D-lysine
hR=homoarginine
_=position not occupied
Aib=amino isobutyric acid
CH₂=methylene
OMe=methoxy
Nle=Nor-leucine
NMe=N-methyl attached to the alpha amino group of an amino acid, e.g., NMeA=N-methyl alanine, NMeV=N-methyl valine
Orn=ornithine
K(CO(CH₂)₂SH)=ε-(3′-mercaptopropionyl)-lysine
K(W)=ε-(L-tryptophyl)-lysine
Abu=α-amino-n-butyric acid or 2-aminobutanoic acid
Cit=citrulline
Dab=diaminobutyric acid
K(Ac)=ε-acetyl lysine
PEG=polyethylene glycol
PEG40K=40,000 Dalton PEG molecule
PEG30K=30,000 Dalton PEG molecule
PEG20K=20,000 Dalton PEG molecule
K(E-C₁₆)=(ε-(γ-L-glutamyl(N-α-palmitoyl))-lysine
[symbol] =a lactam bridge or a disulfide bridge

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochem Biophys Res Commun, 170:643-648 (1990)). The sequences for VIP, PACAP-27, and PACAP-38 are as follows:

| | Seq.ID | |
|---|---|---|
| Peptide | # | Sequence |
| VIP | SEQ ID NO: 3 | HSDAVFTDNYTRLRKQMAVKKYLNSILN |
| PACAP-27 | SEQ ID NO: 4 | HSDGIFTDSYSRYRKQMAVKKYLAAVL- |
| | | NH₂ |
| PACAP-38 | SEQ ID NO: 5 | HSDGIFTDSYSRYRKQMAVKKYLAAVLG |
| | | KRYQRVKNK-NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), and other amino acids having an extra methylene in the side chain (“homo” amino acids), and modified amino acids (e.g norleucine, lysine (isopropyl)—wherein the side chain amine of lysine is modified by an isopropyl group). Also included are amino acids such as ornithine, amino isobutyric acid and amino butanoic acid.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity and by a ratio of VPAC2 receptor binding affinity to PAC1 receptor binding affinity. Binding affinity is determined as described below in Example 4.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT), intraperitoneal glucose tolerance test (IPGTT), and oral glucose tolerance test (OGTT)). Insulinotropic activity is routinely measured in humans by measuring insulin levels or C-peptide levels. Selective VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using two different assays: DiscoveRx and Alpha Screen. See Examples 3 and 5 for further details of these assays. Whilst these assays are performed in different ways, the results demonstrate a general correlation between the two assays.

The term “plasma half-life” refers to the time in which half of the relevant molecules circulate in the plasma prior to being cleared. An alternatively used term is “elimination half-life.” The term “extended” or “longer” used in the context of plasma half-life or elimination half-life indicates there is a statistically significant increase in the half-life of a PEGylated VPAC2 receptor peptide agonist relative to that of the reference molecule (e.g., the non-PEGylated form of the peptide or the native peptide) as determined under comparable conditions. The half-life reported herein is the elimination half-life; it is that which corresponds to the terminal log-linear rate of elimination. The person skilled in the art appreciates that half-life is a derived parameter that changes as a function of both clearance and volume of distribution.

Clearance is the measure of the body's ability to eliminate a drug. As clearance decreases due, for example, to modifications to a drug, half-life would be expected to increase. However, this reciprocal relationship is exact only when there is no change in the volume of distribution. A useful approximate relationship between the terminal log-linear half-life (t_{1/2}), clearance (C), and volume of distribution (V) is given by the equation: t_{1/2}≈0.693 (V/C). Clearance does not indicate how much drug is being removed but, rather, the volume of biological fluid such as blood or plasma that would have to be completely freed of drug to account for the elimination. Clearance is expressed as a volume per unit of time.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected.

The reference peptide used for the percentage sequence identity calculations herein is:

| | |
|---|---|
| P603 | C6- |
| | HSDAVFTEQY(OMe)TOrnLRAibQLAAbuAibOrnYAibQAibI- |
| | OrnOrnGGPSSGAPPPK(E-C16)-NH₂ |

Percent sequence identity may be calculated by determining the number of residues that differ between a peptide encompassed by the present invention and a reference peptide such as P603 (SEQ ID NO: 7), taking that number and dividing it by the number of amino acids in the reference peptide (e.g. 39 amino acids for P603), multiplying the result by 100, and subtracting that resulting number from 100. For example, a sequence having 39 amino acids with four amino acids that are different from P603 would have a percent (%) sequence identity of 90% (e.g. 100−((4/39)×100)). For a sequence that is longer than 39 amino acids, the number of residues that differ from the P603 sequence will include the additional amino acids over 39 for purposes of the aforementioned calculation. For example, a sequence having 40 amino acids, with four amino acids different from the 39 amino acids in the P603 sequence and with one additional amino acid at the carboxy terminus which is not present in the P603 sequence, would have a total of five amino acids that differ from P603. Thus, this sequence would have a percent (%) sequence identity of 87% (e.g. 100−((5/39)×100)). The degree of sequence identity may be determined using methods well known in the art (see, for example, Wilbur, W. J. and Lipman, D. J., Proc. Natl. Acad. Sci. USA 80:726-730 (1983) and Myers E. and Miller W., Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 22(22):4673-4680 (1994)) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The sequence for a selective VPAC2 receptor peptide agonist of the present invention is selective for the VPAC2 receptor and preferably has a sequence identity in the range of 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97%, with P603 (SEQ ID NO: 7). Preferably, the sequence has a sequence identity of greater than 82% with P603 (SEQ ID NO: 7). More preferably, the sequence has greater than 90% sequence identity with P603 (SEQ ID NO: 7). Even more preferably, the sequence has greater than 92% sequence identity with P603 (SEQ ID NO: 7). Yet more preferably, the sequence has greater than 95% sequence identity or 97% sequence identity with P603 (SEQ ID NO: 7).

The term “C₁-C₁₆ alkyl” as used herein means a monovalent saturated straight, branched or cyclic chain hydrocarbon radical having from 1 to 16 carbon atoms or when cyclic, having from 3 to 16 carbon atoms. Thus the term “C₁-C₁₆ alkyl” includes, for example, methyl, ethyl, n-propyl, isopropyl, n-butyl, isobutyl, sec-butyl, tert-butyl, n-heptyl, n-octyl, cyclopropyl, cyclobutyl, cyclopentyl and cyclohexyl. The C₁-C₁₆ alkyl group may be optionally substituted with one or more substituents including, for example, aryl, C₁-C₆ alkoxy, —OH, halogen, —CF₃ and —SH.

The term “C₁-C₆ alkyl” as used herein means a monovalent saturated straight, branched or cyclic chain hydrocarbon radical having from 1 to 6 carbon atoms or when cyclic, having from 3 to 6 carbon atoms. Thus the term “C₁-C₆ alkyl” includes, for example, methyl, ethyl, n-propyl, isopropyl, n-butyl, isobutyl, sec-butyl, tert-butyl, cyclopropyl, cyclobutyl, cyclopentyl and cyclohexyl. The C₁-C₆ alkyl group may be optionally substituted with one or more substituents.

The term “C₂-C₆ alkenyl” as used herein means a monovalent straight, branched or cyclic chain hydrocarbon radical having at least one double bond and having from 2 to 6 carbon atoms or when cyclic, having from 3 to 6 carbon atoms. Thus the term “C₂-C₆ alkenyl” includes vinyl, prop-2-enyl, but-3-enyl, pent-4-enyl and isopropenyl. The C₂-C₆ alkenyl group may be optionally substituted with one or more substituents.

The term “C₂-C₆ alkynyl” as used herein means a monovalent straight or branched chain hydrocarbon radical having at least one triple bond and having from 2 to 6 carbon atoms. Thus the term “C₂-C₆ alkynyl” includes prop-2-ynyl, but-3-ynyl and pent-4-ynyl. The C₂-C₆ alkynyl may be optionally substituted with one or more substituents.

The term “C₁-C₆ alkoxy” as used herein means a monovalent unsubstituted saturated straight-chain or branched-chain hydrocarbon radical having from 1 to 6 carbon atoms linked to the point of substitution by a divalent O radical. Thus the term “C₁-C₆ alkoxy” includes, for example, methoxy, ethoxy, n-propoxy, isopropoxy, n-butoxy, isobutoxy, sec-butoxy and tert-butoxy. The C₁-C₆ alkoxy group may be optionally substituted with one or more substituents.

The term “halo” or “halogen” means fluorine, chlorine, bromine or iodine.

The term “aryl” when used alone or as part of a group is a 5 to 10 membered aromatic or heteroaromatic group including a phenyl group, a 5 or 6-membered monocyclic heteroaromatic group, each member of which may be optionally substituted with 1, 2, 3, 4 or 5 substituents (depending upon the number of available substitution positions), a naphthyl group or an 8-, 9- or 10-membered bicyclic heteroaromatic group, each member of which may be optionally substituted with 1, 2, 3, 4, 5 or 6 substituents (depending on the number of available substitution positions). Within this definition of aryl, suitable substitutions include C₁-C₆ alkyl, C₂-C₆ alkenyl, C₂-C₆ alkynyl, amino, hydroxy, halogen, —SH and CF₃.

The term “arylC₁-C₄ alkyl” as used herein means a C₁-C₄ alkyl group substituted with an aryl. Thus the term “arylC₁-C₄ alkyl” includes benzyl, 1-phenylethyl (α-methylbenzyl), 2-phenylethyl, 1-naphthalenemethyl or 2-naphthalenemethyl.

The term “naphthyl” includes 1-naphthyl, and 2-naphthyl. 1-naphthyl is preferred.

The term “benzyl” as used herein means a monovalent unsubstituted phenyl radical linked to the point of substitution by a —CH₂— group.

The term “5- or 6-membered monocyclic heteroaromatic group” as used herein means a monocyclic aromatic group with a total of 5 or 6 atoms in the ring wherein from 1 to 4 of those atoms are each independently selected from N, O and S. Preferred groups have 1 or 2 atoms in the ring which are each independently selected from N, O and S. Examples of 5-membered monocyclic heteroaromatic groups include pyrrolyl (also called azolyl), furanyl, thienyl, pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. Examples of 6-membered monocyclic heteroaromatic groups include pyridinyl, pyrimidyl, pyrazinyl, pyridazinyl and triazinyl.

The term “8-, 9- or 10-membered bicyclic heteroaromatic group” as used herein means a fused bicyclic aromatic group with a total of 8, 9 or 10 atoms in the ring system wherein from 1 to 4 of those atoms are each independently selected from N, O and S. Preferred groups have from 1 to 3 atoms in the ring system which are each independently selected from N, O and S. Suitable 8-membered bicyclic heteroaromatic groups include imidazo[2,1-b][1,3]thiazolyl, thieno[3,2-b]thienyl, thieno[2,3-d][1,3]thiazolyl and thieno[2,3-d]imidazolyl. Suitable 9-membered bicyclic heteroaromatic groups include indolyl, isoindolyl, benzofuranyl (also called benzo[b]furanyl), isobenzofuranyl (also called benzo[c]furanyl), benzothienyl (also called benzo[b]thienyl), isobenzothienyl (also called benzo[c]thienyl), indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl and imidazo[1,2-a]pyridine. Suitable 10-membered bicyclic heteroaromatic groups include quinolinyl, isoquinolinyl, cinnolinyl, quinazolinyl, quinoxalinyl, 1,5-naphthyridyl, 1,6-naphthyridyl, 1,7-naphthyridyl and 1,8-naphthyridyl.

The term “PEG” as used herein means a polyethylene glycol molecule. In its typical form, PEG is a linear polymer with terminal hydroxyl groups and has the formula HO—CH₂CH₂—(CH₂CH₂O)n-CH₂CH₂—OH, where n is from about 8 to about 4000. The terminal hydrogen may be substituted with a protective group such as an alkyl or alkanol group. Preferably, PEG has at least one hydroxy group, more preferably it is a terminal hydroxy group. It is this hydroxy group which is preferably activated to react with the peptide. There are many forms of PEG useful for the present invention. Numerous derivatives of PEG exist in the art and are suitable for use in the invention. (See, e.g., U.S. Pat. Nos. 5,445,090; 5,900,461; 5,932,462; 6,436,386; 6,448,369; 6,437,025; 6,448,369; 6,495,659; 6,515,100 and 6,514,491 and Zalipsky, S. Bioconjugate Chem. 6:150-165, 1995). The PEG molecule covalently attached to VPAC2 receptor peptide agonists in the present invention is not intended to be limited to a particular type. The molecular weight of the PEG molecule is preferably from 500-100,000 daltons. PEG may be linear or branched and PEGylated VPAC2 receptor peptide agonists may have one, two or three PEG molecules attached to the peptide. It is more preferable that there be one or two PEG molecules per PEGylated VPAC2 receptor peptide agonist, however, when there is more than one PEG molecule per peptide molecule, it is preferred that there be no more than three. It is further contemplated that both ends of the PEG molecule may be homo- or hetero-functionalized for crosslinking two or more VPAC2 receptor peptide agonists together. Where there are two PEG molecules present, the PEG molecules will preferably each be 20,000 dalton PEG molecules or each be 30,000 dalton molecules. However, PEG molecules having a different molecular weight may be used, for example, one 10,000 dalton PEG molecule and one 30,000 PEG molecule, or one 20,000 dalton PEG molecule and one 40,000 dalton PEG molecule.

A PEG molecule may be covalently attached to a Cys or Lys residue. A PEG molecule may also be covalently attached to a Trp residue which is coupled to the side chain of a Lys residue (K(W)). Alternatively, a K(CO(CH₂)₂SH) group may be PEGylated to form K(CO(CH₂)₂S-PEG). Any Lys residue in the peptide agonist may be substituted for a K(W) or K(CO(CH₂)₂SH), which may then be PEGylated. In addition, any Cys residue in the peptide agonist may be substituted for a modified cysteine residue, for example, hC. The modified Cys residue may be covalently attached to a PEG molecule.

The term “PEGylation” as used herein means the covalent attachment of one or more PEG molecules as described above to the VPAC2 receptor peptide agonists of the present invention.

The term “lactam bridge” as used herein means a covalent bond, in particular an amide bond, linking the side chain amino terminus of one amino acid in the peptide agonist to the side chain carboxy terminus of another amino acid in the peptide agonist. Preferably, the lactam bridge is formed by the covalent attachment of the side chain of a residue at Xaaₙ to the side chain of a residue at Xaaₙ₊₄, wherein n is 1 to 28. Also preferably, the lactam bridge is formed by the covalent attachment of the side chain amino terminus of a Lys or Orn residue to the side chain carboxy terminus of an Asp or Glu residue.

The term “disulfide bridge” as used herein means a covalent bond linking a sulfur atom at the side chain terminus of one amino acid in the peptide agonist to a sulfur atom at the side chain terminus of another amino acid in the peptide agonist. Preferably, the disulfide bridge is formed by the covalent attachment of the side chain of a residue at Xaaₙ to the side chain of a residue at Xaaₙ₊₄, wherein n is 1 to 28. Also preferably, the disulfide bridge is formed by the covalent attachment of the side chain of a Cys or hC residue to the side chain of another Cys or hC residue.

According to a preferred embodiment of the present invention, there is provided a VPAC2 receptor peptide agonist comprising an amino acid sequence of Formula 1 (SEQ ID NO: 1) or Formula 2 (SEQ ID NO: 2) wherein Xaa₃ is Asp or Glu, Xaa₈ is Asp or Glu, Xaa₉ is Asn or Gln, Xaa₁₀ is Tyr or Tyr(OMe), Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, Orn, or Arg, Xaa₁₆ is Gln or Lys, Xaa₁₇ is Val, Leu, Ala, Ile, Lys, or Nle, Xaa₁₉ is Ala or Abu, Xaa₂₀ is Lys, Val, Leu, Aib, Ala, Gln, or Arg, Xaa₂₁ is Lys, Aib, Orn, Ala, Gln, or Arg, Xaa₂₃ is Leu or Aib, Xaa₂₅ is Ser or Aib, Xaa₂₇ is Lys, Orn, hR, or Arg, Xaa₂₈ is Asn, Gln, Lys, hR, Aib, Orn, or Pro and/or Xaa₂₉ is Lys, Orn, hR, or is absent, a C-terminal extension comprising the sequence: GGPSSGAPPPK (E-C₁₆), and an N-terminal modification which modification is the addition of hexanoyl or acetyl.

According to another preferred embodiment of the present invention, there is provided a VPAC2 receptor peptide agonist comprising an amino acid sequence of Formula 1 (SEQ ID NO: 1) or Formula 2 (SEQ ID NO: 2) wherein Xaa₈ is Glu, Xaa₉ is Gln, Xaa₁₀ is Tyr(OMe), Xaa₁₂ is Orn, Xaa₁₅ is Aib, Xaa₁₉ is Abu, Xaa₂₀ is Aib, Xaa₂₁ is Orn, Xaa₂₃ is Aib, Xaa₂₅ is Aib, Xaa₂₇ is Orn, and/or Xaa₂₈ is Orn, a C-terminal extension comprising the sequence: GGPSSGAPPPK (E-C₁₆), and an N-terminal modification which modification is the addition of hexanoyl or acetyl.

According to yet another preferred embodiment of the present invention, there is provided a VPAC2 receptor peptide agonist comprising an amino acid sequence of Formula 2 (SEQ ID NO: 2), a C-terminal extension comprising the sequence: GGPSSGAPPPK (E-C₁₆), and an N-terminal modification which modification is the addition of hexanoyl or acetyl.

The present invention is based on the finding that the addition of a C-terminal extension comprising the sequence: GGPSSGAPPPK (E-C₁₆) to the C-terminus of a peptide sequence according to Formula 1 or Formula 2 provides features that may protect the peptide as well as may enhance activity, selectivity, and/or potency. For example, the C-terminal extension may stabilize the helical structure of the peptide and stabilize sites located near to the C-terminus, which are prone to enzymatic cleavage. Furthermore, the C-terminally extended peptides disclosed herein may be more selective for the VPAC2 receptor and can be more potent than VIP, PACAP, and other known VPAC2 receptor peptide agonists.

PEGylation of proteins may overcome many of the pharmacological and toxicological/immunological problems associated with using peptides or proteins as therapeutics. However, for any individual peptide it is uncertain whether the PEGylated form of the peptide will have significant loss in bioactivity as compared to the unPEGylated form of the peptide.

The bioactivity of PEGylated proteins can be affected by factors such as: i) the size of the PEG molecule; ii) the particular sites of attachment; iii) the degree of modification; iv) adverse coupling conditions; v) whether a linker is used for attachment or whether the polymer is directly attached; vi) generation of harmful co-products; vii) damage inflicted by the activated polymer; or viii) retention of charge. Work performed on the PEGylation of cytokines, for example, shows the effect PEGylation may have. Depending on the coupling reaction used, polymer modification of cytokines has resulted in dramatic reductions in bioactivity. [Francis, G. E., et al., (1998) PEGylation of cytokines and other therapeutic proteins and peptides: the importance of biological optimization of coupling techniques, Intl. J. Hem. 68: 1-18]. Maintaining the bioactivity of PEGylated peptides is even more problematic than for proteins. As peptides are smaller than proteins, modification by PEGylation may potentially have a greater effect on bioactivity.

The VPAC2 receptor peptide agonists of the present invention may be modified by the covalent attachment of one or more molecules of PEG. PEGylated peptides generally have improved pharmacokinetic profiles due to slower proteolytic degradation and renal clearance. PEGylation will increase the apparent size of the VPAC2 receptor peptide agonists, thus reducing renal filtration and altering biodistribution. PEGylation can shield antigenic epitopes of the VPAC2 receptor peptide agonists, thus reducing reticuloendothelial clearance and recognition by the immune system and also reducing degradation by proteolytic enzymes, such as DPP-IV.

Covalent attachment of one or more molecules of PEG to a small, biologically active VPAC2 receptor peptide agonist poses the risk of adversely affecting the agonist, for example, by destabilising the inherent secondary structure and bioactive conformation and reducing bioactivity, so as to make the agonist unsuitable for use as a therapeutic. Covalent attachment of one or more molecules of PEG to particular residues of a VPAC2 receptor peptide agonist surprisingly results in a biologically active, PEGylated VPAC2 receptor peptide agonist with an extended half-life and reduced clearance when compared to that of non-PEGylated VPAC2 receptor peptide agonists.

In order to determine the potential PEGylation sites in a VPAC2 receptor peptide agonist, serine scanning may be conducted. A Ser residue is substituted at a particular position in the peptide and the Ser-modified peptide is tested for potency and selectivity. If the Ser substitution has minimal impact on potency and the Ser-modified peptide is selective for the VPAC2 receptor, the Ser residue is then substituted for a Cys or Lys residue, which serves as a direct or indirect PEGylation site. Indirect PEGylation of a residue is the PEGylation of a chemical group or residue which is bonded to the PEGylation site residue. Indirect PEGylation of Lys includes PEGylation of K(W) and K(CO(CH₂)₂SH).

The invention described herein provides VPAC2 receptor peptide agonists which may be covalently attached to one or more molecules of PEG, or a derivative thereof wherein each PEG may be attached to a Cys or Lys amino acid, to a K(W) or a K(CO(CH₂)₂SH) in the peptide agonist. PEGylation can enhance the half-life of the selective VPAC2 receptor peptide agonists, resulting in VPAC2 receptor peptide agonists with an elimination half-life of at least one hour, preferably at least 3, 5, 7, 10, 15, 20, or 24 hours and most preferably at least 48 hours. PEGylated VPAC2 receptor peptide agonists preferably have a clearance value of 200 ml/h/kg or less, more preferably 180, 150, 120, 100, 80, 60 ml/h/kg or less and most preferably less than 50, 40 or 20 ml/h/kg.

The region of wild-type VIP from aspartic acid at position 8 to isoleucine at position 26 has an alpha-helix structure. Increasing the helical content of a peptide enhances potency and selectivity whilst at the same time improving protection from enzymatic degradation. The use of a C-terminal extension may enhance the helicity of the peptide. In addition, the introduction of a covalent bond, for example a lactam bridge, linking the side chains of two amino acids on the surface of the helix, also enhances the helicity of the peptide.

It has furthermore been discovered that modification of the N-terminus of the VPAC2 receptor peptide agonist may enhance potency and/or provide stability against DPP-IV cleavage.

VIP and some known VPAC2 receptor peptide agonists are susceptible to cleavage by various enzymes and, thus, have a short in vivo half-life. Various enzymatic cleavage sites in the VPAC2 receptor peptide agonists are discussed below. The cleavage sites are discussed relative to the amino acid positions in VIP (SEQ ID NO: 3), and are applicable to the sequences noted herein.

Cleavage of the peptide agonist by the enzyme dipeptidyl-peptidase-IV (DPP-IV) occurs between position 2 (serine in VIP) and position 3 (aspartic acid in VIP). The agonists of the present invention may be rendered more stable to DPP-IV cleavage in this region by the addition of a N-terminal modification. Examples of N-terminal modifications that may improve stability against DPP-IV cleavage include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid, or —C(═NH₂)—NH₂. Preferably, the N-terminal modification is the addition of acetyl or hexanoyl.

There are chymotrypsin cleavage sites in wild-type VIP between the amino acids 10 and 11 (tyrosine and threonine) and those at 22 and 23 (tyrosine and leucine). Making substitutions at position 10 and/or 11 and position 22 and/or 23 may increase the stability of the peptide at these sites. For example, substitution of tyrosine at position 10 and/or position 22 with Tyr(OMe) may increase stability. A lactam bridge, for example, linking the side chains of the amino acids at positions 21 and 25 may protect the 22-23 site from cleavage.

There is a trypsin cleavage site between the amino acids at positions 12 and 13 of wild-type VIP. Certain amino acids render the peptide less susceptible to cleavage at this site, for example, ornithine at position 12.

In wild-type VIP, and in numerous VPAC2 receptor peptide agonists known in the art, there are cleavage sites between the basic amino acids at positions 14 and 15 and between those at positions 20 and 21. The selective VPAC2 receptor peptide agonists of the present invention may have improved proteolytic stability in-vivo due to substitutions at these sites. The preferred substitutions at these sites are those which render the peptide less susceptible to cleavage by trypsin-like enzymes, including trypsin. For example, amino isobutyric acid at position 15, amino isobutyric acid at position 20, and ornithine at position 21 are all preferred substitutions which may lead to improved stability.

There is also a cleavage site between the amino acids at positions 25 and 26 of wild type VIP. This cleavage site may be completely or partially eliminated through substitution of the amino acid at position 25 and/or the amino acid at position 26.

The region of the VPAC2 receptor peptide agonist encompassing the amino acids at positions 27, 28 and 29 is also susceptible to enzyme cleavage. The addition of a C-terminal extension may render the peptide agonist more stable against neuroendopeptidase (NEP), it may also increase selectivity for the VPAC2 receptor. This region may also be attacked by trypsin-like enzymes. If that occurs, the peptide agonist may lose its C-terminal extension with the additional carboxypeptidase activity leading to an inactive form of the peptide. Resistance to cleavage in this region may be increased by substituting the amino acid at position 27, 28 and/or 29 with ornithine.

In addition to selective VPAC2 receptor peptide agonists with resistance to cleavage by various peptidases, the selective VPAC2 peptide receptor agonists of the present invention may also encompass peptides with enhanced selectivity for the VPAC2 receptor, increased potency, and/or increased stability compared with some peptides known in the art.

Preferably, selective non-PEGylated VPAC2 receptor peptide agonists have an EC₅₀ value less than 2 nM. More preferably, the EC₅₀ value is less than 1 nM. Even more preferably, the EC₅₀ is less than 0.5 nM. Still more preferably, the EC₅₀ value is less than 0.1 nM. Preferably, selective PEGylated VPAC2 receptor peptide agonists have an EC₅₀ value less than 200 nM. More preferably, the EC₅₀ value is less than 50 nM. Even more preferably, the EC₅₀ value is less than 30 nM. Still more preferably, the EC₅₀ value is less than 10 nM.

Example 4 describes assays for determining selectivity as a ratio of VPAC2 receptor binding affinity to VPAC1 receptor binding affinity and as a ratio of VPAC2 receptor binding affinity to PAC1 receptor binding affinity. Preferably, the agonists of the present invention have a selectivity ratio where the affinity for the VPAC2 receptor is at least 50 times greater than for the VPAC1 and/or for PAC1 receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the ratio is at least 1000 times greater for VPAC2 than for VPAC1 and/or for PAC1.

As used herein, “selective VPAC2 receptor peptide agonists” also include pharmaceutically acceptable salts of the compounds described herein. A selective VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective VPAC2 receptor peptide agonists of the present invention are preferably formulated as pharmaceutical compositions. Standard pharmaceutical formulation techniques may be employed such as those described in Remington's Pharmaceutical Sciences, Mack Publishing Company, Easton, Pa. The selective VPAC2 receptor peptide agonists of the present invention may be formulated for administration through the buccal, topical, oral, transdermal, nasal, or pulmonary route, or for parenteral administration.

Parenteral administration can include, for example, systemic administration, such as by intramuscular, intravenous, subcutaneous, intradermal, or intraperitoneal injection. The selective VPAC2 receptor peptide agonists can be administered to the subject in conjunction with an acceptable pharmaceutical carrier, diluent, or excipient as part of a pharmaceutical composition for treating NIDDM, or the disorders discussed below. The pharmaceutical composition can be a solution or, if administered parenterally, a suspension of the VPAC2 receptor peptide agonist or a suspension of the VPAC2 receptor peptide agonist complexed with a divalent metal cation such as zinc. Suitable pharmaceutical carriers may contain inert ingredients which do not interact with the peptide or peptide derivative. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

The VPAC2 receptor peptide agonists of the invention may be formulated for administration such that blood plasma levels are maintained in the efficacious range for extended time periods. The main barrier to effective oral peptide drug delivery is poor bioavailability due to degradation of peptides by acids and enzymes, poor absorption through epithelial membranes, and transition of peptides to an insoluble form after exposure to the acidic pH environment in the digestive tract. Oral delivery systems for peptides such as those encompassed by the present invention are known in the art. For example, VPAC2 receptor peptide agonists can be encapsulated using microspheres and then delivered orally. For example, VPAC2 receptor peptide agonists can be encapsulated into microspheres composed of a commercially available, biocompatible, biodegradable polymer, poly(lactide-co-glycolide)-COOH and olive oil as a filler (see Joseph, et al. Diabetologia 43:1319-1328 (2000)). Other types of microsphere technology is also available commercially such as Medisorb® and Prolease® biodegradable polymers from Alkermes. Medisorb® polymers can be produced with any of the lactide isomers. Lactide:glycolide ratios can be varied between 0:100 and 100:0 allowing for a broad range of polymer properties. This allows for the design of delivery systems and implantable devices with resorption times ranging from weeks to months. Emisphere has also published numerous articles discussing oral delivery technology for peptides and proteins. For example, see WO 95/28838 by Leone-bay et al. which discloses specific carriers comprised of modified amino acids to facilitate absorption.

The selective VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favourably to VPAC2 receptor stimulation or to the administration of VPAC2 receptor peptide agonists can therefore be treated with the VPAC2 agonists of the present invention. These subjects are said to “be in need of treatment with VPAC2 agonists” or “in need of VPAC2 receptor stimulation”.

The selective VPAC2 receptor peptide agonists of the present invention may be employed to treat diabetes, including both type 1 and type 2 diabetes (non-insulin dependent diabetes mellitus or NIDDM). The agonists may also be used to treat subjects requiring prophylactic treatment with a VPAC2 receptor agonist, e.g., subjects at risk for developing NIDDM. Such treatment may also delay the onset of diabetes and diabetic complications. Additional subjects which may be treated with the agonists of the present invention include those with impaired glucose tolerance (IGT) (Expert Committee on Classification of Diabetes Mellitus, Diabetes Care 22 (Supp. 1):S5, 1999) or impaired fasting glucose (IFG) (Charles, et al., Diabetes 40:796, 1991), subjects whose body weight is about 25% above normal body weight for the subject's height and body build, subjects having one or more parents with NIDDM, subjects who have had gestational diabetes, and subjects with metabolic disorders such as those resulting from decreased endogenous insulin secretion. The selective VPAC2 receptor peptide agonists may be used to prevent subjects with impaired glucose tolerance from proceeding to develop NIDDM, prevent pancreatic β-cell deterioration, induce β-cell proliferation, improve β-cell function, activate dormant β-cells, differentiate cells into β-cells, stimulate β-cell replication, and inhibit β-cell apoptosis. Other diseases and conditions that may be treated or prevented using agonists of the invention in methods of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); gestational diabetes (Metzger, Diabetes, 40:197, 1991); metabolic syndrome X, dyslipidemia, hyperglycemia, hyperinsulinemia, hypertriglyceridemia, and insulin resistance.

The selective VPAC2 receptor peptide agonists of the invention may also be used to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenyloin, thyroid hormone, β-adrenergic agents, α-interferon and drugs used to treat HIV infection.

The selective VPAC2 receptor peptide agonists of the present invention may be effective in the suppression of food intake and the treatment of obesity.

The selective VPAC2 receptor peptide agonists of the present invention may also be effective in the prevention or treatment of such disorders as atherosclerotic disease, hyperlipidemia, hypercholesteremia, low HDL levels, hypertension, primary pulmonary hypertension, cardiovascular disease (including atherosclerosis, coronary heart disease, and coronary artery disease), cerebrovascular disease and peripheral vessel disease; and for the treatment of lupus, polycystic ovary syndrome, carcinogenesis, and hyperplasia, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

In addition, the selective VPAC2 receptor peptide agonists of the invention may be used for treatment of asthma (Bolin, et al., Biopolymer 37:57-66 (1995); U.S. Pat. No. 5,677,419; showing that polypeptide R3PO is active in relaxing guinea pig tracheal smooth muscle); hypotension induction (VIP induces hypotension, tachycardia, and facial flushing in asthmatic patients (Morice, et al., Peptides 7:279-280 (1986); Morice, et al., Lancet 2:1225-1227 (1983)); for the treatment of male reproduction problems (Siow, et al., Arch. Androl. 43(1):67-71 (1999)); as an anti-apoptosis/neuroprotective agent (Brenneman, et al., Ann. N.Y. Acad. Sci. 865:207-12 (1998)); for cardioprotection during ischemic events (Kalfin, et al., J. Pharmacol. Exp. Ther. 1268(2):952-8 (1994); Das, et al., Ann. N.Y. Acad. Sci. 865:297-308 (1998)); for manipulation of the circadian clock and its associated disorders (Hamar, et al., Cell 109:497-508 (2002); Shen, et al., Proc. Natl. Acad. Sci. 97:11575-80, (2000)); as an anti-ulcer agent (Tuncel, et al., Ann. N.Y. Acad. Sci. 865:309-22, (1998)), and as a treatment for AIDS (Branch, et al., Blood, 106: Abstract 1427, (2005)).

An “effective amount” of a selective VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective VPAC2 receptor peptide agonist administered to a subject will also depend on the type and severity of the disease and on the characteristics of the subject, such as general health, age, sex, body weight and tolerance to drugs. The dose of selective VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective VPAC2 receptor peptide agonists of the present invention can be prepared by using standard methods of solid-phase peptide synthesis techniques. Peptide synthesizers are commercially available from, for example, Rainin-PTI Symphony Peptide Synthesizer (Tucson, Ariz.). Reagents for solid phase synthesis are commercially available, for example, from Glycopep (Chicago, Ill.). Solid phase peptide synthesizers can be used according to manufacturers instructions for blocking interfering groups, protecting the amino acid to be reacted, coupling, decoupling, and capping of unreacted amino acids.

Typically, an α-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991. Examples include t-butyloxycarbonyl (tBoc) and fluorenylmethoxycarbonyl (Fmoc).

The selective VPAC2 receptor peptide agonists may also be synthesized using standard automated solid-phase synthesis protocols using t-butoxycarbonyl- or fluorenylmethoxycarbonyl-alpha-amino acids with appropriate side-chain protection. After completion of synthesis, modification of the N-terminus may be accomplished by reacting the α-amino group with, for example: (i) active esters (using similar protocols as described above for the introduction of an α-N-protected amino acid); (ii) aldehydes in the presence of a reducing agent (reductive amination procedure); and (iii) guanidation reagents. Then, peptides are cleaved from the solid-phase support with simultaneous side-chain deprotection using standard hydrogen fluoride methods or trifluoroacetic acid (TFA). Crude peptides are then further purified using Reversed-Phase Chromatography on VYDAC C18 columns using acetonitrile gradients in 0.1% TFA. To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

Once a peptide of the present invention is prepared and purified, it may be modified by covalently linking one or more PEG molecules to Cys, Lys, K(W) or K(CO(CH₂)₂SH) residues in the peptide. A wide variety of methods have been described in the art to produce peptides covalently conjugated to PEG and the specific method used for the present invention is not intended to be limiting (for review article see, Roberts, M. et al. Advanced Drug Delivery Reviews, 54:459-476, 2002).

An example of a PEG molecule which may be used is methoxy-PEG2-MAL-40K, a bifurcated PEG maleimide (Nektar, Huntsville, Ala.). Other examples include, but are not limited to bulk mPEG-SBA-20K (Nektar), mPEG2-ALD-40K (Nektar), and methoxy-PEG-MAL-30K (Dow).

One method for preparing VPAC2 receptor peptide agonists involves the use of PEG-maleimide to directly attach PEG to a thiol group of the peptide. The introduction of a thiol functionality can be achieved by adding or inserting a Cys or hC residue onto or into the peptide at positions described above. A thiol functionality can also be introduced onto the side-chain of the peptide (e.g. acylation of lysine ε-amino group by a thiol-containing acid, such as mercaptopropionic acid). A PEGylation process of the present invention utilizes Michael addition to form a stable thioether linker. The reaction is highly specific and takes place under mild conditions in the presence of other functional groups. PEG maleimide has been used as a reactive polymer for preparing well-defined, bioactive PEG-protein conjugates. It is preferable that the procedure uses a molar excess, preferably from 1 to 10 molar excess, of a thiol-containing VPAC2 receptor peptide agonist relative to PEG maleimide to drive the reaction to completion. The reactions are preferably performed between pH 4.0 and 9.0 at room temperature for 10 minutes to 40 hours. The excess of unPEGylated thiol-containing peptide is readily separated from the PEGylated product by conventional separation methods. The VPAC2 receptor peptide agonist is preferably isolated using reverse-phase HPLC or size exclusion chromatography. Specific conditions required for PEGylation of VPAC2 receptor peptide agonists are set forth in Example 8. Cysteine PEGylation may be performed using PEG maleimide or bifurcated PEG maleimide.

An alternative method for PEGylating VPAC2 receptor peptide agonists involves PEGylating a lysine residue using a PEG-succinimidyl derivative. In order to achieve site specific PEGylation, the Lys residues which are not used for PEGylation may be substituted for Arg residues.

Another approach for PEGylation is via Pictet-Spengler reaction. A Trp residue with its free amine is needed to incorporate the PEG molecule onto a VPAC2 receptor selective peptide. One approach to achieve this is to site specifically introduce a Trp residue onto the amine of a Lys sidechain via an amide bond during the solid phase synthesis (see Example 10).

The cyclisation of a VPAC2 receptor peptide agonist may be carried out in solution or on a solid support. Cyclisation on a solid support can be performed immediately following solid phase synthesis of the peptide. This involves the selective or orthogonal protection of the amino acids which will be covalently linked in the cyclisation.

Various preferred features and embodiments of the present invention will now be described with reference to the following non-limiting examples.

### EXAMPLE 1

### Preparation of the Selective VPAC2 Receptor Peptide Agonists by Solid Phase t-Boc Chemistry

Approximately 0.5-0.6 grams (0.38-0.45 mmole) Boc Ser(Bzl)-PAM resin is placed in a standard 60 mL reaction vessel. Double couplings are run on an Applied Biosystems ABI430A peptide synthesizer. The following side-chain protected amino acids (2 mmole cartridges of Boc amino acids) are obtained from Midwest Biotech (Fishers, Ind.) and are used in the synthesis:

Arg-tosyl (Tos), Asp-cyclohexyl ester (OcHx), Asp-9-fluorenylmethyl (Fm), Cys-p-methylbenzyl (p-MeBzl), Glu-cyclohexyl ester (OcHx), His-benzyloxymethyl(Bom), Lys-2-chlorobenzyloxycarbonyl (2Cl-Z), Lys-9-fluorenylmethoxycarbonyl (Fmoc), Orn-2-chlorobenzyloxycarbonyl (2Cl-Z), Ser-O-benzyl ether (OBzl), Thr-O-benzyl ether (OBzl), Trp-formyl (CHO), Tyr-2-bromobenzyloxycarbonyl (2Br-Z), Boc-Ser(OBzl) PAM resin, and MBHA resin. Trifluoroacetic acid (TFA), di-isopropylethylamine (DIEA), 1.0 M hydroxybenzotriazole (HOBt) in NMP and 1.0 M dicyclohexylcarbodiimide (DCC) in NMP are purchased from PE-Applied Biosystems (Foster City, Calif.). Dimethylformamide (DMF-Burdick and Jackson) and dichloromethane (DCM-Mallinkrodt) is purchased from Mays Chemical Co. (Indianapolis, Ind.). Benzotriazole-1-yl-oxy-tris-(dimethylamino)-phosphoniumhexafluorophosphate (BOP) is obtained from NovaBiochem (San Diego, Calif.).

Standard double couplings are run using either symmetric anhydride or HOBt esters, both formed using DCC. At the completion of the syntheses, the N-terminal Boc group is removed and the peptidyl resins are treated with 20% piperidine in DMF to deformylate the Trp side chain if Trp is present in the sequence. For the N-terminal acylation, four-fold excess of symmetric anhydride of the corresponding acid is added onto the peptide resin. The symmetric anhydride is prepared by diisopropylcarbodiimde (DIC) activation in DCM. The reaction is allowed to proceed for 4 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25 cm VYDAC C18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

Cyclic VPAC2 receptor peptide agonists with a lactam bridge linking a lysine residue and an aspartic acid residue may be prepared by selectively protecting the side chains of the lysine and the aspartic acid residue with Fmoc and Fm, respectively. All other amino acids used in the synthesis are standard benzyl side-chain protected Boc-amino acids. Cyclisation may then be carried out on the solid support immediately following solid phase synthesis of the peptide. The Fmoc and Fm protecting groups are selectively removed and the cyclisation is carried out by activating the aspartic acid carboxyl group with BOP in the presence of DIEA. The reaction is allowed to proceed for 24 hours and monitored by ninhydrin test.

### EXAMPLE 2

### Preparation of the Selective VPAC2 Receptor Peptide Agonists by Solid Phase FMoc Chemistry

Approximately 114 mg (50 mMole) FMOC Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere. Tuebingen, Germany).

The following Fmoc amino acids are purchased from GlycoPep (Chicago, Ill.), and NovaBiochem (La Jolla, Calif.): Arg-2,2,4,6,7-pentamethyldihydrobenzofuran-5-sulfonyl (Pbf), Asn-trityl (Trt), Asp-β-t-Butyl ester (tBu), Asp-β-allyl ester (Allyl), Glu-6-t-butyl ester (tBu), Glu-δ-allyl ester (Allyl), Gln-trityl (Trt), His-trityl (Trt), Lys-t-butyloxycarbonyl (Boc), Lys-allyloxycarbonyl (Aloc), Orn-allyloxycarbonyl (Aloc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone (NMP-Burdick and Jackson), dichloromethane (DCM-Mallinkrodt) are purchased from Mays Chemical Co. (Indianapolis, Ind.).

Hydroxybenzotrizole (HOBt), di-isopropylcarbodiimide (DIC), di-isopropylethylamine (DIEA), and piperidine (Pip) are purchased from Aldrich Chemical Co (Milwaukee, Wis.). Benzotriazole-1-yl-oxy-tris-(dimethylamino)-phosphoniumhexafluorophosphate (BOP) is obtained from NovaBiochem (San Diego, Calif.).

All amino acids are dissolved in 0.3 M in DMF. Three hour DIC/HOBt activated couplings are run after 20 minutes deprotection using 20% Piperidine/DMF. Each resin is washed with DMF after deprotections and couplings. After the last coupling and deprotection, the peptidyl resins are washed with DCM and are dried in vacuo in the reaction vessel. For the N-terminal acylation, four-fold excess of symmetric anhydride of the corresponding acid is added onto the peptide resin. The symmetric anhydride is prepared by DIC activation in DCM. The reaction is allowed to proceed for 4 hours and monitored by ninhydrin test. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 mL TFA, all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo.

0.3-0.6 mg of each product is dissolved in 1 mL 0.1% TFA/acetonitrile (ACN), with 20 μL being analyzed on HPLC [0.46×15 cm METASIL AQ C18, 1 mL/min, 45 C.°, 214 nM (0.2 A), A=0.1% TFA, B=0.1% TFA/50% ACN. Gradient=50% B to 90% B over 30 minutes].

Purifications are run on a 2.2×25 cm VYDAC C18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0 A) and collecting 1 minute fractions. Appropriate fractions are combined, frozen and lyophilized. Dried products are analyzed by HPLC (0.46×15 cm METASIL AQ C18) and MALDI mass spectrometry.

Cyclic VPAC2 receptor peptide agonists with a lactam bridge linking a lysine residue and an aspartic acid residue are prepared by selectively protecting the side chains of the lysine residue and the aspartic acid residue with Aloc and Allyl, respectively. All other amino acids used in the synthesis are standard t-Butyl side chain protected Fmoc-amino acids.

Cyclisation may then be carried out on the solid support immediately following solid phase synthesis of the peptide. The Aloc and Allyl protecting groups are selectively removed and the cyclisation is carried out by activating the aspartic acid carboxyl group with BOP in the presence of DIEA.

### Preparation of P603 by Solid-Phase Fmoc Chemistry:

Approximately 75 mg (50 μMols) of polystyrene Rink Amide AM resin (Rapp Polymere GmbH, Tubingen, Germany) is placed in a reaction vessel. Fmoc-Lys-allyloxycarbonyl (Aloc) is used in the first synthetic cycle of the automated synthesis using a Rainin Symphony Peptide Synthesizer. The elongation of the peptide resin is carried out as described above in Example 2. After completion of the automated elongation of the peptide-resin including C6-N-terminal acylation, the Aloc protecting group is removed manually using Tetrakis(triphenylphosphine) palladium (0) [100 μMols] in DCM-acetic acid-piperidine (92:5:3, v/v/v) (Aldrich Chemical Co., Milwaukee, Wis.) for 20 min at 25° C. This step is repeated twice. The aloc deprotected resin is then washed with 5% DIEA in DCM and 0.03 M sodium diethyldithiocarbamate trihydrate (Aldrich Chemical Co., Milwaukee, Wis.) in DMF. Fmoc-Glu-α-OtBu ester (500 μMols; purchased from NovaBiochem, La Jolla, Calif.) is incorporated manually using DIC (500 μMols) and HOBt (500 μMols) in DMF for 2 hours at 25° C. After subsequent Fmoc removal, palmitic acid (500 μMols; purchased from Aldrich Chemical Co., Milwaukee, Wis.) is incorporated using the same method as for Fmoc-Glu-α-OtBu ester. Cleavage of the peptide from the resin and purification are carried out as described in Example 2.

### EXAMPLE 3

### In-Vitro Potency at Human VPAC2 Receptors

Alpha screen: Cells (CHO-S cells stably expressing human VPAC2 receptors) are washed in the culture flask once with PBS. Then, the cells are rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate. Results for each agonist are, at minimum, from two analyses performed in a single run. For some agonists, the results are the mean of more than one run. The tested peptide concentrations are: 10000, 1000, 100, 10, 3, 1, 0.1, 0.01, 0.003, 0.001, 0.0001 and 0.00001 nM.

DiscoveRx: A CHO-S cell line stably expressing human VPAC2 receptor in a 96-well microtiter plate is seeded with 50,000 cells/well the day before the assay. The cells are allowed to attach for 24 hours in 200 μL culture medium. On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. The typically tested concentrations of peptide are: 1000, 300, 100, 10, 1, 0.3, 0.1, 0.01, 0.001, 0.0001 and 0 nM.

The activity (EC₅₀ (nM)) for the human VPAC2 receptors is reported in Table 1 for the different assay formats.

TABLE 1
Peptide potency at human VPAC2 receptors

VPAC2 (cAMP)

| Agonist # | Alpha | DiscoveRx |
|---|---|---|
| VIP | 1.0 | 0.7 |
| Pacap-27 | 2.3 | 0.8 |
| P603 | n.d. | 0.26 |

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled PACAP-27 for VPAC1, VPAC2 and PAC1 as the tracer.

For this assay, the solutions and equipment include:

Presoak solution: 0.5% Polyethyleneamine in Aqua dest

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate Multiscreen FB Opaque Plate; 1.0 μM Type B Glasfiber filter

In order to prepare the filter plates, the presoak solution is aspirated by vacuum filtration. The plates are flushed twice with 200 μL flush buffer. 200 μL blocking buffer is added to the filter plate. The filter plate is then incubated with 200 μL presoak solution for 1 hour at room temperature.

The assay plate is filled with 25 μL assay buffer, 25 μL membranes (2.5 μg) suspended in assay buffer, 25 μL compound (agonist) in assay buffer, and 25 μL tracer (about 40000 cpm) in assay buffer. The filled plate is incubated for 1 hour with shaking.

The transfer from assay plate to filter plate is conducted. The blocking buffer is aspirated by vacuum filtration and washed two times with flush buffer. 90 μL is transferred from the assay plate to the filter plate. The 90 μL transferred from assay plate is aspirated and washed three times with 200 μL flush buffer. The plastic support is removed. It is dried for 1 hour at 60° C. 30 μL Microscint is added. The count is performed.

### EXAMPLE 5

### In Vitro Potency at Rat VPAC1 and VPAC2 Receptors

DiscoveRx: CHO-PO cells are transiently transfected with rat VPAC1 or VPAC2 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay is performed.

On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. The typically tested concentrations of peptide are: 1000, 300, 100, 10, 1, 0.3, 0.1, 0.01, 0.001, 0.0001 and 0 nM.

### EXAMPLE 6

### In Vivo Assays

Intravenous glucose tolerance test (IVGTT): Normal Wistar rats are fasted overnight and are anesthetized prior to the experiment. A blood sampling catheter is inserted into the rats. The agonist is given subcutaneously, normally 24 h prior to the glucose challenge. Blood samples are taken from the carotid artery. A blood sample is drawn immediately prior to the injection of glucose along with the agonist. After the initial blood sample, glucose mixed is injected intravenously (i.v.). A glucose challenge of 0.5 g/kg body weight is given, injecting a total of 1.5 mL vehicle with glucose and agonist per kg body weight. The peptide concentrations are varied to produce the desired dose in μg/kg. Blood samples are drawn at 2, 4, 6 and 10 minutes after giving glucose. The control group of animals receives the same vehicle along with glucose, but with no agonists added. In some instances, 20 and 30 minute post-glucose blood samples were drawn. Aprotinin is added to the blood sample (250-500 kIU/ml blood). The plasma is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known. Variability in the maximal response is mostly due to variability in the vehicle dose. Protocol details are as follows:

| | |
|---|---|
| SPECIES/STRAIN/WEIGHT | Rat/Wistar Unilever/approximately 275-300 g |
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to surgery. |
| LIVE-PHASE PARAMETERS | Animals are sacrificed at the end of the test. |
| IVGTT: Performed on rats (with two | Glucose IV bolus: 500 mg/kg as 10% |
| catheters, jugular vein and carotid | solution (5 mL/kg) at time = 0. |
| artery) of each group, under pentobarbital | Compound iv: 0-240 min prior to glucose |
| anesthesia. | Blood samplings (300 μL from carotid artery; |
| | EDTA as anticoagulant; aprotinin and PMSF |
| | as antiproteolytics; kept on ice): 0, 2, 4, 6, and |
| | 10, 20 and 30 minutes. |
| | Parameters determined: Insulin + glucose |
| TOXICOKINETICS | Plasma samples remaining after insulin |
| | measurements are kept at −20° C. and |
| | compound levels are determined. |

### EXAMPLE 7

### Rat Serum Stability Studies

In order to determine the stability of VPAC2 receptor peptide agonists in rat serum, CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit are obtained. The rat serum is stored at 4° C. until use and is used within two weeks.

On Day 0, two 100 μL aliquots of 10 μM peptide in rat serum are prepared by adding 10 μL peptide stock to 90 μL rat serum for each aliquot. 250 kIU aprotinin/mL is added to one of these aliquots. The aliquot is stored with aprotinin at 4° C. The aliquot is stored without aprotinin at 37° C. The aliquots are incubated for 24 hours.

On Day 1, after incubation of the aliquots prepared on day 0 for 24 hours, an incubation buffer containing PBS+1.3 mM CaCl₂, 1.2 mM MgCl₂, 2 mM glucose, and 0.5 mM IBMX is prepared. A plate with 11 serial 3× dilutions of peptide in serum for the 4° C. and 37° C. aliquot is prepared for each peptide studied. 4000 nM is used as the maximal concentration. The plate(s) with cells are washed twice in incubation buffer and the cells are incubated in 50 μL incubation media per well for 15 minutes. 50 μL solution per well is transferred to the cells from the plate prepared with 11 serial 3× dilutions of peptide for the 4° C. and 37° C. aliquot for each peptide studied, using the maximal concentrations that are indicated by the primary screen, in duplicate. This step dilutes the peptide concentration by a factor of two. The cells are incubated at room temperature for 30 minutes. The supernatant is removed. 40 μL/well of the DiscoveRx antibody/extraction buffer is added. The cells are incubated on the shaker (300 rpm) for 1 hour. Normal procedure with the DiscoveRx kit is followed. cAMP standards are included in column 12. EC₅₀ values are determined from the cAMP assay data. The remaining amount of active peptide is estimated by the formula EC_{50, 4C}/EC_{50, 37C} for each condition.

TABLE 5
Estimated peptide stability after 24 h in rat serum at 37° C.

| Agonist # | % stab¹ |
|---|---|
| P603 | 67 |

¹Values >100% may represent release of intact peptide from the PEG conjugate

### EXAMPLE 8

### PEGylation of Selective VPAC2 Receptor Peptide Agonists Using Thiol-Based Chemistry

In general, PEGylation reactions are run under conditions that permit the formation of a thioether bond. Specifically, the pH of the solution ranges from about 4 to 9 and the thiol-containing peptide concentrations range from 0.7 to 10 molar excess of PEG maleimide concentration. The PEGylation reactions are normally run at room temperature. The VPAC2 receptor peptide agonist is then isolated using reverse-phase HPLC or size exclusion chromatography (SEC). PEGylated peptide analogues are characterized using analytical RP-HPLC, HPLC-SEC, SDS-PAGE, and/or MALDI Mass Spectrometry.

Usually a thiol function is introduced into or onto a selective VPAC2 receptor peptide agonist by adding a cysteine or a homocysteine or a thiol-containing moiety at either or both termini or by inserting a cysteine or a homocysteine or a thiol-containing moiety into the sequence. Thiol-containing VPAC2 receptor peptide agonists are reacted with 40 kDa, 30 kDa or 20 kDa PEG-maleimide to produce derivatives with PEG covalently attached via a thioether bond.

### EXAMPLE 9

### PEGylation Via Acylation on the Sidechain of Lysine

In order to achieve site-specific PEGylation of selective VPAC2 receptor peptide agonists, all the Lys residues are changed into Arg residues except for Lys residues where PEGylation is intended. A PEG molecule which may be used is mPEG-SBA-20K (Nektar, Lot #: PT-04E-11). The PEGylation reaction is preferably performed at room temperature for 2-3 hours. The peptide is purified by preparative HPLC.

### EXAMPLE 10

### PEGylation Via Pictet-Spengler Reaction

For PEGylation via Pictet-Spengler reaction to occur, a Trp residue with its free amine is needed to incorporate the PEG molecule onto the selective VPAC2 receptor peptide agonist. One approach to achieve this is to couple a Trp residue onto the sidechain of Lys. The extensive SAR indicates that this modification does not change the properties of the parent peptide in terms of its in vitro potency and selectivity.

PEG with a functional aldehyde, for example mPEG2-BUTYRALD-40K (Nektar, USA), is used for the reaction. The site specific PEGylation involves the formation a tetracarboline ring between PEG and the peptide. PEGylation is conducted in glacial acetic acid at room temperature for 1 to 48 hours. A 1 to 10 molar excess of the PEG aldehyde is used in the reaction. After the removal of acetic acid, the VPAC2 receptor peptide agonist is isolated by preparative RP-HPLC.

Other modifications of the present invention will be apparent to those skilled in the art without departing from the scope of the invention.

1. A VPAC2 receptor peptide agonist comprising the amino acid sequence shown in SEQ ID NO: 1:
(SEQ ID NO: 1)
Xaa₁-Xaa₂₋Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-
Xaa₁₉-Xaa₂₀-Xaa₂₁-Xaa₂₂-Xaa₂₃-Xaa₂₄-Xaa₂₅-Xaa₂₆-
Xaa₂₇-Xaa₂₈-Xaa₂₉-Xaa₃₀-Xaa₃₁-Xaa₃₂
Formula 1
wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Glu, Ser, Cys, or K(CO(CH₂)₂SH);
Xaa₁₀ is: Tyr, Trp, or Tyr(OMe);
Xaa₁₂ is: Arg, Lys, hR, Orn, Aib, Ala, Leu, Gln, Phe, or Cys;
Xaa₁₃ is: Leu, Phe, Glu, Ala, Aib, Ser, Cys, or K(CO(CH₂)₂SH);
Xaa₁₄ is: Arg, Leu, Lys, Ala, hR, Orn, Phe, Gln, Aib, or Cit;
Xaa₁₅ is: Lys, Ala, Arg, Glu, Leu, Orn, Phe, Gln, Aib, K(Ac), Cys, K(W), or K(CO(CH₂)₂SH);
Xaa₁₆ is: Gln, Lys, Ala, Ser, Cys, or K(CO(CH₂)₂SH);
Xaa₁₇ is: Val, Ala, Leu, Ile, Met, Nle, Lys, Aib, Ser, Cys, K(CO(CH₂)₂SH), or K(W);
Xaa₁₈ is: Ala, Ser, Cys, or Abu;
Xaa₁₉ is: Ala, Leu, Gly, Ser, Cys, K(CO(CH₂)₂SH), or Abu;
Xaa₂₀ is: Lys, Gln, hR, Arg, Ser, Orn, Ala, Aib, Trp, Thr, Leu, Ile, Phe, Tyr, Val, K(Ac), Cys, or K(CO(CH₂)₂SH);
Xaa₂₁ is: Lys, Arg, Ala, Phe, Aib, Leu, Gln, Orn, hR, K(Ac), Ser, Cys, K(W), K(CO(CH₂)₂SH), or hC;
Xaa₂₂ is: Tyr, Trp, Phe, Thr, Leu, Ile, Val, Tyr(OMe), Ala, Aib, or Ser;
Xaa₂₃ is: Leu, Phe, Ile, Ala, Trp, Thr, Val, Aib, or Ser;
Xaa₂₄ is: Gln, Asn, Ser, Cys, K(CO(CH₂)₂SH), or K(W);
Xaa₂₅ is: Ser, Asp, Phe, Ile, Leu, Thr, Val, Trp, Gln, Asn, Tyr, Aib, Glu, Cys, K(CO(CH₂)₂SH), or hC;
Xaa₂₆ is: Ile, Leu, Thr, Val, Trp, Tyr, Phe, Aib, Ser, Cys, K(CO(CH₂)₂SH), or K(W);
Xaa₂₇ is: Lys, hR, Arg, Gln, Orn, or dK;
Xaa₂₈ is: Asn, Gln, Lys, Arg, Aib, Orn, hR, Pro, dK, Cys, K(CO(CH₂)₂SH), or K(W);
Xaa₂₉ is: Lys, Ser, Arg, Asn, hR, Cys, Orn, or is absent;
Xaa₃₀ is: Arg, Lys, Ile, hR, or is absent;
Xaa₃₁ is: Tyr, His, Phe, Gln, or is absent; and
Xaa₃₂ is: Cys, or is absent;
provided that if Xaa₂₉, Xaa₃₀, Xaa₃₁, or Xaa₃₂ is absent, the next amino acid present downstream is the next amino acid in the peptide agonist sequence;
and a C-terminal extension comprising the amino acid sequence:
GGPSSGAPPPK(E-C₁₆)
wherein said C-terminal amino acid may be amidated.

2-10. (canceled)

11. The VPAC2 receptor peptide agonist according to claim 1, wherein said agonist is PEGylated.

12. The VPAC2 receptor peptide agonist according to claim 1, wherein said agonist is cyclic.

13. The VPAC2 receptor peptide agonist according to claim 1, further comprising an N-terminal modification at the N-terminus of said peptide agonist, wherein said N-terminal modification is selected from the group consisting of:
(a) addition of D-histidine, isoleucine, methionine, or norleucine;
(b) addition of a peptide comprising the amino acid sequence Ser-Trp-Cys-Glu-Pro-Gly-Trp-Cys-Arg (SEQ ID NO: 6) wherein said Arg is linked to the N-terminus of said peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; an aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; arylC₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃, arylC₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, arylC₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

14. The VPAC2 receptor peptide agonist according to claim 13, wherein said N-terminal modification is the addition of a group selected from the group consisting of: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂.

15. The VPAC2 receptor peptide agonist according to claim 14, wherein said N-terminal modification is the addition of acetyl or hexanoyl.

16. The VPAC2 receptor peptide agonist according to claim 1, comprising the amino acid sequence C6-HSDAVFTEQY(OMe)TOrnLRAibQLAAbuAibOrnYAibQAibIOrnOrnGGPSSGAPPPK(E-C16)-NH₂ (SEQ ID NO: 7).

17. A pharmaceutical composition, comprising a VPAC2 receptor peptide agonist according to claim 1 and one or more pharmaceutically acceptable diluents, carriers or excipients.

18-20. (canceled)

21. A method of treating non-insulin-dependent diabetes or insulin-dependent diabetes, or of suppressing food intake, in a patient in need thereof, comprising administering to said patient an effective amount of a VPAC2 receptor peptide agonist according to claim 1.

22-23. (canceled)
